(19) United States
(12) Patent Application Publication
(10) Pub. No.: US 2024/0225907 A1
(43) Pub. Date: Jul. 11, 2024

(54) Method and Device for Impregnating a Film, and Method for Manufacturing a Transdermal Therapeutic System

(71) Applicant: LTS Lohmann Therapie-Systeme AG, Andernach (DE)

(72) Inventors: Peter Steinborn, Neuwied (DE); Georg Schlögel, Andernach (DE); Dorothea Gutekunst, Montabaur (DE); Viktoria Steinbrecher, Andernach (DE); Gerd Hoffmann, Neuwied (DE); Sandra Wiedersberg, Steigra (DE)

(21) Appl. No.: 18/572,041

(22) PCT Filed: May 31, 2022
(86) PCT No.: PCT/EP2022/064713

§ 371 Date: Dec. 19, 2023

(30) Foreign Application Priority Data
Jun. 21, 2021 (DE) 10 2021 115 977.6

(51) Int. Cl. A61F 13/00; B05C 3/12

(52) U.S. Cl. CPC A61F 13/00991; A61F 13/00063; B05C 3/125; B05D 2500/00

(57) ABSTRACT

A method for impregnating a film, including the following steps feeding the film to be impregnated, immersing the film into an impregnating agent, and partially separating the impregnating agent from the film in order to achieve a predefined loading of the film with impregnating agent. Furthermore, a device for impregnating a film by means of an immersion basin filled with impregnating agent, a film guiding, and a separating device for separating the impregnating agent from the film in order to achieve a predefined loading of the film with impregnating agent. Furthermore, a method for manufacturing a transdermal therapeutic system.

### CROSS REFERENCE TO RELATED APPLICATIONS

This application is the United States national phase of International Patent Application No. PCT/EP2022/064713 filed May 31, 2022, and claims priority to German Patent Application No. 10 2021 115 977.6 filed Jun. 21, 2021, the disclosures of which are hereby incorporated by reference in their entireties.

### BACKGROUND OF THE INVENTION

### Field of the Invention

The invention relates to a method for impregnating a film, a device for impregnating a film, and a method for manufacturing a transdermal therapeutic system.

### Description of Related Art

Impregnated films, in particular membranes, are used as a layer in transdermal therapeutic systems, for example.

For example, U.S. Pat. No. 4,436,741 A describes a transdermal therapeutic system for the delivery of scopolamine comprising an oil-impregnated membrane.

A common method for impregnating films, such as membranes, is coating on a coating machine using reverse roll coating with an engraving wave.

When using engraving rollers, the amount of impregnation is determined by the engraving of the roller, e.g. the engraving gap. The roller, e.g. the engraving depth, must be changed for any variation in the amount of impregnation.

A disadvantage of the existing method is, among other things, the inaccurate loading of the film with impregnating agent.

Furthermore, previous methods are time-consuming in terms of production, complex and/or require additional work steps and/or installations.

### SUMMARY OF THE INVENTION

It is any object of the present invention to provide a method for impregnating a film, a device for impregnating a film, and a method for manufacturing a transdermal therapeutic system, wherein the impregnation is optimized.

According to the invention, the object of the present invention is achieved by a method to impregnating a film as described herein, a device for impregnating a film as described herein, and a method for manufacturing a transdermal therapeutic system as described herein.

The method for impregnating a film according to the invention is in particular a method for impregnating a membrane, preferably for use in transdermal therapeutic systems. A first step of the method is to feed the film to be impregnated. Preferably, feeding takes place in a rolling process so that the film is unwound from a feed roller. The fed film is immersed in an impregnating agent. In particular, the film is completely immersed in the impregnating agent. The impregnating agent is preferably arranged in an immersion basin, also known as an impregnating bath. In a preferred embodiment, the film is guided, at least partially, substantially horizontally through the impregnating agent. It is preferred that the film is fully loaded with impregnating agent after passing through the impregnating agent. The impregnating agent is then partially separated from the film. This separation takes place in particular in such a way that a predefined loading of the film with impregnating agent is achieved. In particular, the separation results in the film being loaded with oil from 5 g/m² to 20 g/m², preferably from 7 g/m² to 13 g/m², particularly preferably from approx. 10 g/m². It is preferred that the separation consists of wiping the impregnating agent off the film.

The device for impregnating a film according to the invention is in particular a device for impregnating a membrane, preferably for use in transdermal therapeutic systems. The device has an immersion basin filled with impregnating agent, also known as an impregnating bath. The immersion basin is configured and/or arranged for immersing the film into the impregnating agent, in particular in a continuous manner. The device has a film guiding. The film guiding is configured in particular for feeding the film, for passing the film through, for immersing the film into the immersion basin and/or for feeding the film out. Feeding the film means in particular guiding the film into the device, passing the film through means in particular guiding the film within the device, immersing the film means in particular guiding the film through the immersion basin, and feeding the film out means in particular guiding the film out of the device. It is preferred that at least one, in particular rotatable deflection roller is arranged at least partially within the immersion basin to guide the film. It is particularly preferred that at least two, in particular exactly two, deflection rollers are arranged within the immersion basin such that the film can be guided substantially horizontally, at least through a part of the immersion basin. Furthermore, it is preferred that the film guiding has at least one, in particular rotatable, roller, e.g. a deflection roller. The at least one roller is preferably designed to be round. It is preferred that at least one roller is driven. Preferably, the film guiding has a feed roller for unwinding the film and/or an out-feed roller for winding the film. In particular, at least one of the feed roller and the out-feed roller are configured to be driven. The at least one roller, in particular the feed roller and/or the out-feed roller, can preferably be arranged outside, inside or partially outside and inside the device. Moreover, the device comprises a separating device for separating, in particular partially, the impregnating agent from the film. The separating device preferably is configured such that the impregnating agent is separated to achieve a predefined loading of the film with impregnating agent.

In the method according to the invention, it is preferred that the separation of the impregnating agent is effected by means of a separating device. The separating device used within the scope of the method is configured in particular like the separating device of the device according to the invention.

In a preferred embodiment, the separating device comprises at least one wiper. The wiper preferably has a silicone coating. In particular, the wiper is coated, preferably laminated, with a silicone film of preferably 0.8 mm to 3 mm thickness. It is preferred that the wiper is configured and/or arranged such that it comes into contact with the impregnating agent, preferably is pressed against it. In particular, the wiper is pressurized. It is preferred that the separating device separates the impregnating agent on one side of the film or on both sides of the film.

In a preferred embodiment, the separating device comprises at least one edge and/or at least one, preferably rotatable, roller. The edge preferably has a square or round structure. It is preferred that the edge is configured and/or arranged such that the edge comes into contact with the incoming or outgoing film relative to the edge. In other words, it is preferred that the edge has an acute or obtuse or right angle to the film in relation to the film running direction. It is preferred that the separating device comprises at least pressure sheet forming the edge. In particular, the pressure plate is bendable.

In a preferred embodiment, the wiper is arranged on both sides of the film loaded with impregnating agent.

In a preferred embodiment, the wiper comes into contact with the film on both sides, in particular by pressing against it. It is preferred that at least one wiper has two opposite rollers in a double-sided configuration. On the other hand, the double-sided wiper can have two opposite edges, or one edge and one opposite pressing surface, e.g. of an anvil.

In a preferred embodiment, the separating device, in particular the at least one wiper, is configured to be variably adjustable so that the predefined loading of the film with impregnating agent can be achieved by separating the impregnating agent. Thus, it is preferred that the separating device, in particular the at least one wiper, is configured such that a desired loading, e.g. a predefined quantity of oil in liters or kilograms per film surface in square meters, can be achieved.

In a preferred embodiment, the separating device comprises at least one adjusting device for adjusting the predefined loading. In particular, the adjusting device has or consists of one of the following devices: a pneumatic device, a hydraulic device, a spring device, an elastomer, a pressure screw and a fine adjustment thread. In particular, said adjusting device is arranged and/or configured to adjust a pressure with which the at least one wiper presses on the film and/or a distance between the at least one wiper and the film. In a double-sided configuration of the at least one wiper, the adjusting device is arranged and/or configured in particular for adjusting a pressure and/or a distance between the two opposite elements, e.g. rollers, edges or pressure surface, of the wiper. In particular, the spring device comprises a compression or tension spring, in particular in the form of a spiral spring. The elastomer, in particular comprising rubber, is in particular an elastic band. It is preferred that an angle of attack of the wiper, in particular in the configuration with an edge, is adjustable. For example, it is preferably implemented that the angle of the edge, e.g. of the pressure sheet, can be changed relative to an opposite edge or to an opposite pressure surface. Due to the changeability of the wiper, in particular different loading conditions are adjustable. It is preferred that the opposite elements of the wiper in double-sided configuration are offset or parallel to each other. It is preferred that an offset of the opposite elements is adjustable.

It is preferred that a separating device comprises one or more of the above-described wipers, in particular in different embodiments.

In a preferred embodiment, the separating device is at least partially configured and/or arranged such that the separated impregnating agent is returned to the immersion basin. Preferably, the separating device is at least partially configured and/or arranged such that the separated impregnating agent falls into to the immersion basin due to gravity. Here it is preferred that the separating device is at least partially arranged above the immersion basin. The method according to the invention has, in particular in addition to or as an alternative to this configuration of the separating device, the step of returning, preferably by gravity, at least parts of the separated impregnating agent. It is preferred that the film is guided substantially vertically, e.g. by the film guiding, after leaving the immersion basin.

In a preferred embodiment, the device comprises a collecting tray, preferably connected to the immersion basin, for collecting the separated impregnating agent. The collecting tray is preferably connected to the immersion basin in a fluid-conducting manner. On the other hand, it is preferred that the collecting tray is configured to be separate from the immersion basin. It is preferred that the collecting tray is larger, in particular in terms of surface area, than the immersion basin. Preferably, the immersion basin and the collecting tray cover the area below the separating device, in particular completely, so that separated impregnating agent, e.g. dripping down, is collected.

In a preferred embodiment, the impregnating agent is an oil, in particular mineral oil. The impregnating agent is particularly kind to the skin.

In a preferred embodiment, the film comprises polypropylene, in particular consists thereof. The film is particularly configured to be porous, preferably microporous. In particular, the film is Celgard® 2400 from the company Celgard.

In a preferred embodiment, the method according to the invention comprises the following further step: performing a test, in particular process control, of the film loaded with impregnating agent. The step is particularly performed after separating the impregnating agent in order to implement the desired loading. In particular, the present loading is determined during testing. Said present loading corresponds in particular to an ACTUAL loading. It is preferred to additionally compare the ACTUAL loading with a TARGET loading. It is preferred that the step of adjusting the separating device is additionally carried out, in particular to adjust the ACTUAL loading to the TARGET loading. The testing is carried out in particular in an optical and/or weight-reducing manner. It is preferred that a part of the film is removed for testing, e.g. cut out, and then said section is tested. The test can preferably be carried out using the testing device described below.

In a preferred embodiment, the device comprises a testing device for testing the film loaded with impregnating agent. The testing device is arranged behind the separating device, in particular in the direction in which the film is guided. The testing device preferably comprises a scale for weighing the film. In addition or alternatively, the testing device has in particular at least one optical control device, such as a camera. The testing device can also be configured to be magnetic or electrical, for example, so that e.g. the conductivity of the film is measurable.

An in particular final step of the method for impregnating a film preferably consists of feeding the impregnated film out. Feeding out is carried out in a rolling process in particular, so that the impregnated film or a film composite comprising the impregnated film is wound onto an out-feed roller. The feed roller and/or the out-feed roller can preferably be driven.

In particular, the features described within the scope for impregnating a film are also applicable to the device, and vice versa. The method for impregnating a film is preferably performed with a device described above.

The method for manufacturing a transdermal therapeutic system according to the invention is in particular a method for manufacturing a transdermal patch or a preproduct for a transdermal patch. The method for manufacturing a transdermal therapeutic system comprises the steps of the method described above for impregnating a film. It is preferred that when performing the method for impregnating a film the device described above is used. The method for manufacturing a transdermal therapeutic system further comprises the step for adding an active ingredient into and/or onto the impregnated film. Here it is possible that the film is coated with the active ingredient, in particular in liquid form. On the other hand or in addition, it is also possible for the impregnated film to be bonded with one or more films containing active ingredients to form a film composite. In particular, the active ingredient comprises scopolamine.

The method for impregnating a film or the method for manufacturing a transdermal therapeutic system is in particular a continuous method.

Within the scope of this application, impregnated means in particular loading a film, in particular a porous film, with an impregnating agent such as oil.

The invention preferably also relates to a device system for manufacturing a transdermal therapeutic system. The device system comprises a device for impregnating a film according to the invention as well as an active ingredient supply device. In particular, the active ingredient supply device is arranged and/or configured for adding an active ingredient into and/or onto the impregnated film. Here it is possible that the film is coated with the active ingredient, in particular in liquid form. On the other hand or in addition, it is also possible for the impregnated film to be bonded with one or more films containing active ingredients to form a film composite. In particular, the active ingredient comprises scopolamine.

Another advantage of the invention is that the work step of impregnation, in particular when the film is used in transdermal therapeutic systems, can be performed within an assembly system or during assembly, instead of as an additional coating work step or within a coating system, as was previously the case in prior art.

In the following, the invention is described in more detail by means of preferred embodiments with reference to the accompanying drawings.

### BRIEF DESCRIPTION OF THE DRAWINGS

The terms Fig., Figs., Figure, and Figures are used interchangeably in the specification to refer to the corresponding figures in the drawings.

In the Figures:

FIG. 1 shows a schematic side view of an embodiment of the device according to the invention,

FIGS. 2a-2c show a schematic illustration of alternative wipers on the basis of the detailed section II of FIG. 1, and

FIG. 3 shows a perspective side view of another embodiment of the device according to the invention.

### DESCRIPTION OF THE DRAWINGS

In the Figures, similar or identical components or elements are identified by the same reference numerals or variations thereof (e.g. 12 and 12′, 12″ or 14 and 14′, 14″).

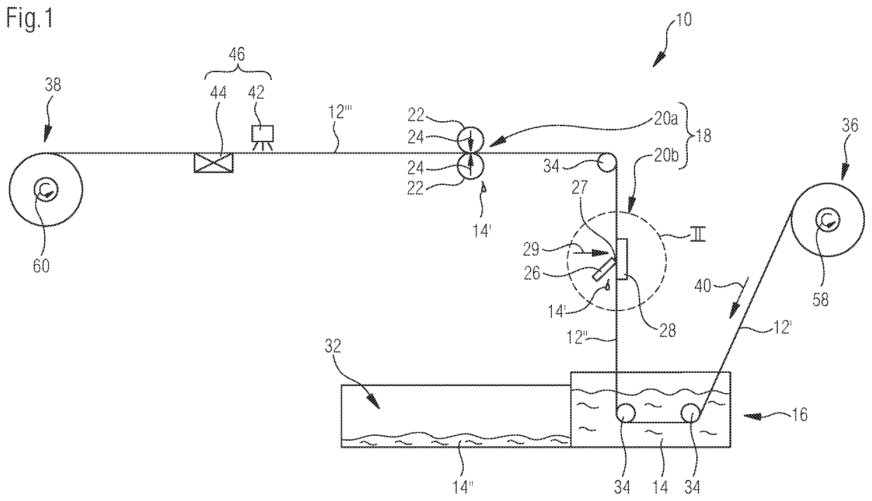

FIG. 1 shows an embodiment of a device 10 for impregnating a film 12 according to the invention.

The preferably porous film 12 is unwound from a feed roller 36, the feed roller 36 rotating counterclockwise, shown by arrow 58. The film 12 is therefore guided in the direction of arrow 40.

The film 12 not yet loaded with impregnating agent 14 is identified by reference numeral 12′, while the loaded film 12 has reference numeral 12″.

The film 12′ is first guided into an immersion basin 16 filled with impregnating agent 14 and loaded there, preferably completely, with impregnating agent 14. Two deflection rollers 34 are arranged inside immersion basin 16 such that film 12 runs substantially horizontally through the impregnating agent 14 in immersion basin 16. The loaded film 12″ then exits immersion basin 16.

The film 12″ is then guided into a first wiper 20b of a separating device 18. Here, film 12″ is pressed against pressure surface 28 by pressure sheet 26 with edge 27 (shown by arrow 29). Parts of impregnating agent 14 are thereby separated from film 12″. This is exemplified by drop 14′ of removed impregnating agent 14. Due to the arrangement of wiper 20b relative to immersion basin 16, the removed impregnating agent 14 falls back into immersion basin 16 and can be reused.

The film 12″ is guided further into another wiper 20a of separating device 18 via deflection roller 34. Said wiper 20a comprises two opposite rollers 22. It is preferred that at least one of the rollers 22, in particular two rollers 22, are pressurized (shown by arrow 24) so that a pressure is applied to film 12″. The wiper 20a further removes impregnating agent 14 from film 12″, exemplified by drop 14′. The separated impregnating agent 14′ falls into a collecting tray 32 collecting impregnating agent 14″.

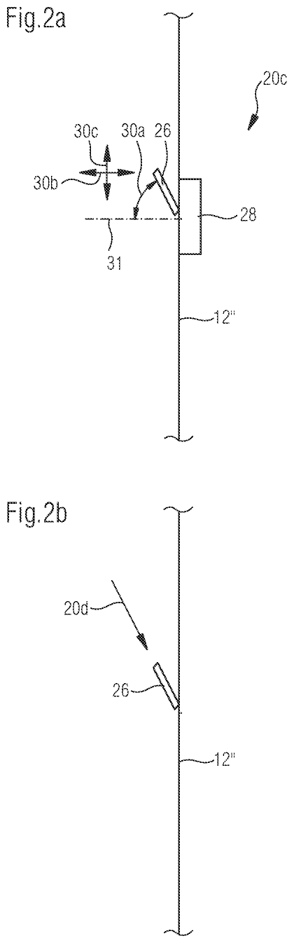

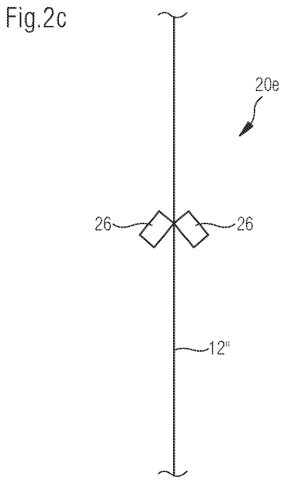

In particular, separating device 18 implements a predefined loading of film 12 with impregnating agent 14. Here, the illustrated configuration of separating device 18 with wiper 20a, 20b is only one preferred possibility. Configurations of separating device 18 with one or more wipers, different wiper configurations (see e.g. FIGS. 2a2c), different arrangements of the wipers, and/or different adjusting devices for the wipers are possible. The adjusting device of the wipers 20a, 20b is only schematically shown in FIG. 1 by the arrows 24 for wiper 20a and by arrow 29 for wiper 20b. It is preferred that the adjusting device is configured to be adjustable. The adjusting device can be a pneumatic device, a hydraulic device, a spring device, an elastomer, a pressure screw and/or a fine adjustment thread, for example. The adjusting device can preferably be used to change the distance and/or the angle and/or the pressure between the wiper elements, shown between the rollers 22 or pressure sheet 26 and pressure surface 28.

The deflection rollers 34 as well as feed roller 36 and out-feed roller 38 are used to implement a film guiding. Here, different variations are also possible. The feed roller 36 and/or out-feed roller 38 can be driven.

After the film has passed the shown wiper 20a, in particular a film 12′″ is present in a desired loading condition.

The film 12′″ is then tested in a testing device 46. In particular, the loading condition of film 12′″ is tested, preferably measured. The testing device 46 is shown with a scale 44 for weighing film 12′″. In particular, a surface section of film 12″″ is weighed. Furthermore, the shown testing device 46 comprises an optical testing device 42, such as a camera. Loading film 12′″ generally changes the appearance of film 12′″. For example, film 12′″ becomes more transparent as the loading of impregnating agent increases. This can be detected by the optical testing device 42. Instead of the shown testing device 46, another configuration is also possible, for example a testing device 46 with only one scale 44 or only one optical testing device 42. An otherwise identical configuration of device 10 is also possible without testing device 46.

Film 12′″ is then wound onto out-feed roller 38. The out-feed roller 38 is shown rotating counterclockwise (arrow 60).

It is possible that an active ingredient, e.g. scopolamine, is bonded with film 12″ before and/or after film 12′″ is wound. On the one hand, a further film layer with active ingredient can be bonded with film 12′″ and/or the active ingredient is bonded directly to film 12′″, e.g. as a coating. Thus, a transdermal therapeutic system can be manufactured.

FIGS. 2a-2c exemplarily show alternative configurations of a wiper 20c, 20d, 20e. Said wipers 20c, 20d, 20e can be used, for example, instead of wiper 20b at position II in FIG. 1.

FIG. 2a shows a wiper 20c having a pressure surface 28 and a pressure sheet 26 arranged opposite. Here, pressure sheet 26 is configured to be adjustable, in particular relative to pressure surface 28. It is shown, for example, that the angle (arrow 30a) of pressure sheet 26 can be changed. For example, an angle between +90° and −90° starting from auxiliary line 31 is possible. An adjustability with an angle between +45° and −45° is preferred. On the other hand, it is shown that also the distance (arrow 30b) between pressure sheet 26 and pressure surface 28 is adjustable. This allows the pressure to be changed, for example. This can be done, for example, by means of a spring device and/or an adjusting screw. Furthermore, it is shown that an offset (arrow 30c) between pressure sheet 26 and pressure surface 28 is adjustable. In alternative, preferred configurations, only one or more of the adjustment possibilities is available. It is preferably also possible to implement the adjustment possibilities in other wiper configurations.

FIG. 2b shows a one-sided wiper 20d having a pressure sheet 26 that is brought into contact with film 12″ and thereby removes impregnating agent.

FIG. 2c shows another embodiment of a wiper 20e, wherein two opposing, obliquely arranged pressure sheets 26 come into contact with film 12″ on both sides and/or with impregnating agent and thus remove the impregnating agent.

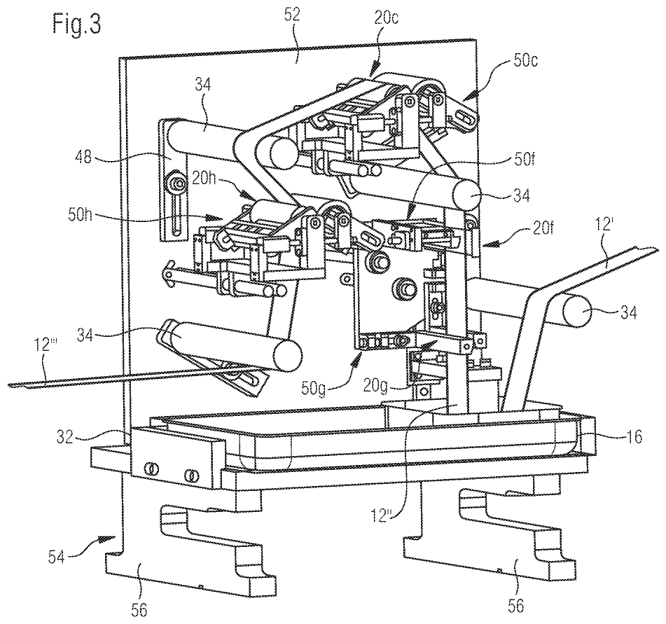

FIG. 3 shows another embodiment of a device 10 for impregnating a film 12 according to the invention. The device of FIG. 3 is at least partially configured in a similar way to device 10 in FIG. 1 (see above).

The device 10 comprises a mounting plate 52 arranged via a standing device 54 having two feet 56, to which at least some of the components of device 10 are attached.

In the figure, film 12′ is fed from the right and film 12′″ is fed out on the left.

The device 10 comprises several wipers 20a-20h of a separating device with which impregnating agent is removed from film 12′.

The wipers 20e, 20h are configured as roller pair wipers, wherein it is preferred that the pressure is adjustable by means of adjusting devices 50e, 50h.

The wiper 20e comprises a pressure sheet 26 and a pressure surface 28, wherein in particular the angle of pressure sheet 26 is variable.

The wiper 20f comprises two pressure sheets 26 which are shown slightly offset. A corresponding arrangement without offset or with a larger offset is also possible. It is also possible that the angle of one or both pressure sheets 26 is variable.

The deflection rollers 34 are variable, in particular by means of adjusting devices 48.

The method for impregnating a film according to the invention and/or the method for manufacturing a transdermal therapeutic system according to the invention can preferably be performed with one or more feature of FIGS. 1-3.

1. A method for impregnating a film, comprising the following steps:
feeding the film to be impregnated;
immersing the film into an impregnating agent preferably arranged in an immersion basin; and
partially separating, in particular wiping off, the impregnating agent from the film in order to achieve a predefined loading of the film with impregnating agent.

2. A device for impregnating a film, comprising:
an immersion basin filled with impregnating agent;
a film guiding for feeding a film to be impregnated, immersing the film into the immersion basin, and feeding the film out; and
a separating device for separating the impregnating agent from the film in order to achieve a predefined loading of the film with impregnating agent.

3. The method according to claim 1, wherein the separation of the impregnating agent is effected by means of a separating device.

4. The method according to claim 3, wherein the separating device comprises at least one wiper, preferably coated with silicone.

5. The method according to claim 4, wherein the wiper comprises at least one edge and/or at least one roller.

6. The method according to claim 4, wherein the in particular pressurized wiper is arranged on both sides of the film loaded with impregnating agent.

7. The method according to claim 3, wherein the separating device is configured to be variably adjustable so that the predefined loading be achieved by separating the impregnating agent.

8. The method according to claim 7 wherein the separating device
comprises at least one adjusting device for adjusting a pressure and/or a distance, in particular of the wiper, the adjusting device preferably comprising: a pneumatic device, a hydraulic device, a spring device, an elastomer, a pressure screw and a fine adjustment thread.

9. The method according to claim 3, wherein the separating device is arranged such that the separated impregnating agent is returned to the immersion basin, in particular falls into the immersion basin.

10. The method according to claim 1 wherein a collecting tray, preferably connected to the immersion basin, for collecting the separated impregnating agent.

11. The method according to claim 1, wherein the impregnating agent comprises oil, preferably mineral oil, in particular consists thereof.

12. The method according to claim 1, wherein the film is a porous film, in particular comprising polypropylene.

13. The method according to claim 1, wherein the method comprises the following further step:
performing in particular an optical and/or weight-recording process control for testing the film loaded with impregnating agent.

14. The method according to claim 1, wherein a testing device, in particular comprising a scale and/or an optical control device, for testing the film loaded with impregnating agent.

15. A method for manufacturing a transdermal therapeutic system, comprising the following steps:
performing the method according to claim 1;
adding an active ingredient into and/or onto the film.
